SEQUENCE LISTING (32 sequences)

SEQ ID NO: 1 - protein, 24 aa, Caenorhabditis elegans
IGYEIGSKLAAMCDDFDAQMMSYS

SEQ ID NO: 2 - protein, 24 aa, Homo sapiens
PEIWIAQELRRIGDEFNAYYARRV

SEQ ID NO: 3 - protein, 24 aa, Homo sapiens
IIRNIARHLAQVGDSMDRSIPPGL

SEQ ID NO: 4 - protein, 24 aa, Homo sapiens
GSDALALRLACIGDEMDVSLRAPR

SEQ ID NO: 5 - protein, 24 aa, Homo sapiens
AAQLTAARLKALGDELHQRTMWRR

SEQ ID NO: 6 - protein, 24 aa, Homo sapiens
WAREIGAQLRRMADDLNAQYERRR

SEQ ID NO: 7 - protein, 24 aa, Homo sapiens
AAQRYGRELRRMSDEFVDSFKKGL

SEQ ID NO: 8 - protein, 24 aa, Homo sapiens
LPPEFAAQLRKIGDKVYCTWSAPD

SEQ ID NO: 9 - protein, 24 aa, Homo sapiens
DLKDECAQLRRIGDKVNLRQKLLN

SEQ ID NO: 10 - protein, 24 aa, Homo sapiens
STKKLSECLKRIGDELDSNMELQR

SEQ ID NO: 11 - protein, 24 aa, Homo sapiens
TMGQVGRQLAIIGDDINRRYDSEF

SEQ ID NO: 12 - protein, 24 aa, Homo sapiens
RLAEVCTVLLRLGDELEQIRPSVY

SEQ ID NO: 13 - protein, 24 aa, Homo sapiens
VTPVVHLTLRQAGDDFSRRYRRDF

SEQ ID NO: 14 - protein, 24 aa, Homo sapiens
PMAAVKQALREAGDEFELRYRRAF

SEQ ID NO: 15 - protein, 24 aa, Homo sapiens
AADPLHQAMRAAGDEFETRFRRTF

SEQ ID NO: 16 - protein, 24 aa, Homo sapiens
TSRKALETLRRVGDGVQRNHETAF

SEQ ID NO: 17 - protein, 24 aa, Caenorhabditis elegans
MRVMGTIFEKKHAENFETFCEQLL

SEQ ID NO: 18 - protein, 24 aa, Homo sapiens
LRQAGDDFSRRYRRDFAEMSRQLH

SEQ ID NO: 19 - protein, 24 aa, Homo sapiens
LREAGDEFELRYRRAFSDLTSQLH

SEQ ID NO: 20 - protein, 24 aa, Homo sapiens
MRAAGDEFETRFRRTFSDLAAQLH

SEQ ID NO: 21 - protein, 24 aa, Homo sapiens
LRRVGDGVQRNHETAFQGMLRKLD

SEQ ID NO: 22 - protein, 22 aa, Homo sapiens
LSECLKRIGDELELQRMIAAVD

SEQ ID NO: 23 - protein, 22 aa, Homo sapiens
VGRQLAIIGDDIMLQHLQPTAE

SEQ ID NO: 24 - protein, 22 aa, Homo sapiens
VCTVLLRLGDELVARQLHIPLQ

SEQ ID NO: 25 - protein, 18 aa, Caenorhabditis elegans
DVVRTVGYGRLIGLISFG

SEQ ID NO: 26 - protein, 18 aa, Homo sapiens
TVVEELFWGRIVAFFEFG

SEQ ID NO: 27 - protein, 18 aa, Homo sapiens
QVVNELFWGRIVAFFSFG

SEQ ID NO: 28 - protein, 18 aa, Homo sapiens
QVSDELFWGRLVAFFVFG

SEQ ID NO: 29 - protein, 18 aa, Homo sapiens
RVMIHVFWGRIVTLISFG

SEQ ID NO: 30 - protein, 18 aa, Homo sapiens
RVAADMFWGRVVALFYFA

SEQ ID NO: 31 - protein, 18 aa, Homo sapiens
KIATSLFWGRVVALLGFG

SEQ ID NO: 32 - protein, 18 aa, Homo sapiens
AVAGHIFWGKVVSLYSAA

(19) United States
(12) Patent Application Publication
(10) Pub. No.: US 2008/0138847 A1
(43) Pub. Date: Jun. 12, 2008

(54) Bcl-2 family member and BH-3 only proteins for use in development of peptidomimetics

(76) Inventor: Yigong Shi, Plainsboro, NJ (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/233,998

(22) Filed: Sep. 23, 2005

Related U.S. Application Data
(60) Provisional application No. 60/612,423, filed on Sep. 23, 2004.

(51) Int. Cl. C12Q 1/02; G01N 33/00; C07K 14/435; C07K 7/00; C12N 5/00

(52) U.S. Cl. 435/29; 436/86; 530/325; 530/326; 435/375

(57) ABSTRACT

Embodiments of the present invention relate to the molecular interactions of Bcl-2 family members. The design of peptidomimetics that discriminate between anti-apoptotic and pro-apoptotic Bcl-2 family members and uses the same to modulate apoptosis are described herein.

### CROSS REFERENCE TO RELATED APPLICATIONS

This application claims the benefit of U.S. Provisional Application No. 60/612,423, filed Sep. 23, 2004 entitled “Structural, Biochemical, and Functional Analyses of CED-9 Recognition by the Proapoptotic Proteins EGL-1 and CED-4,” which is incorporated herein in its entirety by reference thereto.

### FIELD OF THE INVENTION

The present invention analogs or agents that can differentiate between anti-apoptotic and pro-apoptotic Bcl-2 family members so as to modulate programmed cell death (apoptosis). The present invention, generally, relates to methods of identifying and isolating agents that are useful in treating diseases involving apoptosis.

### BACKGROUND

Apoptosis, or programmed cell death, plays a central role in the development and homeostasis of all multi-cellular organisms. Alterations in apoptotic pathways have been implicated in many types of human pathologies, including developmental disorders, cancer, autoimmune diseases, and neuro-degenerative disorders. Apoptotic pathways are attractive targets for development of therapeutic agents. DNA damage, in many cases, activates mitochondria-mediated apoptotic pathways which destroys the affected cell. Radiation and chemotherapy lack molecular specificity and induce apoptosis indiscriminately causing severe side-effects that make these forms of treatment difficult for patients.

Apoptosis is executed primarily by activated caspases, a family of cysteine proteases that cleaves their substrates after an aspartate residue. Caspases are produced as inactive zymogens that are activated by proteolytic processing during apoptosis to become active proteases. Caspase activation is regulated by members of the Bcl-2 family of proteins whose members include both anti-apoptotic and pro-apoptotic proteins. Bcl-2 family members are thought to act by regulating mitochondrial membrane permeability. Pro-apoptotic members of the Bcl-2 superfamily act to increase mitochondrial membrane permeability which allows the release of pro-apoptotic proteins such as Cytochrome-C into the cytoplasm, while anti-apoptotic members counteract this effect.

Bcl-2 family member proteins are characterized by the presence of conserved sequence motifs, specifically, four Bcl-2 homology (BH) domains (BH1, BH2, BH3 and BH4), and can be broken down into three distinct subfamilies. The Bcl-2 subfamily contains anti-apoptotic Bcl-2 family members including Bcl-2, Bcl-xL and Bcl-w. Members of this subfamily contain all four BH domains. Anti-apoptotic Bcl-2 subfamily members are thought to function by sequestering and inactivating pro-apoptotic Bax subfamily members. The Bax subfamily contains pro-apoptotic members and includes Bax and Bak. Members of this subfamily are multi-domain proteins that contain BH-1, 2 and 3 domains and promote apoptosis. The BH-3 Only subfamily have only the conserved BH-3 domain and include Bid, Bim and Bad. The BH-3 Only subfamily members are thought to act as sensors for distinct apoptotic pathways.

Genetic analysis of apoptosis in Caenorhabditis elegans (C. elegans) has identified four genes, egl-1, ced-9, ced-4, and ced-3, that control the death of 131 somatic cells during hermaphrodite development. CED-3 belongs to the caspase family. During apoptosis, the CED-3 zymogen is auto-proteolytically activated by the adaptor molecule CED-4 (an Apaf-1 homologue). However in the absence of an apoptotic stimulus, CED-4 is sequestered by the mitochondria-bound CED-9 and is unable to activate CED-3. CED-9 shares significant sequence homology with the mammalian anti-apoptotic proteins Bcl-2 and Bcl-xL and is a Bcl-2 subfamily member.

The binding of pro-apoptotic protein EGL-1 to CED-9 activates apoptosis. EGL-1 resembles the pro-apoptotic BH-3 Only proteins and is a member of the BH-3 Only subfamily of Bcl-2 proteins. EGL-1, which is transcriptionally activated by upstream apoptotic signals, disrupts the CED-4/CED-9 interaction releasing CED-4 so that it can initiate apoptosis by activating CED-3 caspase. EGL-1's interaction with CED-9 appears to be mediated by the C-terminal 46 amino acids.

Suppression of apoptosis is a contributing factor for a range of diseases including but not limited to developmental disorders, autoimmune diseases, neuro-degenerative disorders and cancer. In particular, overexpression of Bcl-2 and Bcl-xL has been implicated in a number of malignant cancer types. Thus, finding a strategy to specifically target Bcl-2 and Bcl-xL is important to potential anti-cancer therapies.

### SUMMARY

The invention presented herein relates to the field of cell proliferative disease treatment. Specifically, the invention features the description, design and methods of testing of peptides, polypeptides, and peptidomimetics with the ability to bind to specific members of the Bcl-2 family of anti-apoptosis proteins and induce apoptosis. The ability to design ligands that specifically bind to anti-apoptotic Bcl-2 family member proteins and induce apoptosis will allow for the creation of therapeutic agents for the treatment of disorders characterized by disruption of normal apoptotic processes that specifically target injured tissue and reduce side-effects to the patient.

One embodiment of the present invention provides a method of identifying agents that promote apoptosis by selectively interacting with Bcl-2 subfamily member proteins. The method may include the steps of observing binding of an agent with a Bcl-2 subfamily member protein, such as Bcl-2 and Bcl-xL, observing binding of an agent with a Bax subfamily member protein, such as Bax or Bak, and selecting an agent based upon preferential binding with Bcl-2 subfamily member proteins. In a further embodiment, the agent mimics the binding of the C-terminal 45 amino acids of EGL-1 to CED-9, preferable at amino acid residues 54, 55, 58, 61, 62, 63, and 65.

A further embodiment of the present invention is a composition comprising the amino acid sequence N—X₁—X₂—(Xₐₐ)₂—X₃—(Xₐₐ)₂—X₄—X₅—X₆—(Xₐₐ)₁—X₇-M and a carrier, wherein N=0 to 53, M=0 to 26, and wherein said compound binds preferentially to Bcl-2 subfamily member proteins. The composition may bind at a hydrophobic pocket on the surface of Bcl-2 through interactions between amino acids in said pocket and the side-chains of amino acid residues X₁-X₇ of said composition. Preferably, the composition exhibits hydrogen bonding and van der Waals interactions. In further embodiments, the side chains of amino acids X₅, X₆, and X₇ may be modified to fill the aqueous space created by the van der Waals radii of amino acids residues Met119, Phe123, Lys126, Phe133, Gln137, Leu138, Val152, Thr155, Val156, Gly169, Arg170, Gly171, Ile172, Phe177, and Met 231 of CED-9.

Another embodiment of the present invention provides a method of inducing apoptosis comprising administering a compound that selectively binds to Bcl-2, and wherein said compound does not bind to Bax.

Another embodiment of the present invention provides a method of making a compound that selectively binds to Bcl-2 subfamily member proteins. The method may include the steps of constructing a compound that interacts with CED-9, wherein said compound binds to a hydrophobic pocket on the surface of CED-9 and determining whether the compound promotes apoptosis. The compound preferably mimics the binding of the C-terminal 45 amino acids of EGL-1 to CED-9, more preferably at amino acids residues 54, 55, 58, 61, 62, 63, and 65.

A further embodiment of the present invention provides a method of identifying agents that promote cellular proliferation by selectively interacting with Bax subfamily member proteins. The method may include the steps of observing binding of an agent with a Bcl-2 subfamily member protein, such as Bcl-2 and Bcl-xL, observing binding of an agent with a Bax subfamily member protein, such as Bax or Bak, and selecting an agent based upon preferential binding with Bax family member proteins.

Another embodiment of the present invention is an isolated and purified peptide comprising the amino acid sequence N—X₁—X₂—(Xₐₐ)₂—X₃—(Xₐₐ)₂—X₄,—X₅—X₆—(Xₐₐ)₁—X₇-M, wherein N=0 to 53, M=0 to 26, and wherein said compound binds preferentially to Bcl-2 subfamily member proteins.

Another embodiment of the present invention is an isolated and purified peptide comprising the amino acid sequence N—X₁—X₂—(Xₐₐ)₂—X₃—(Xₐₐ)₂—X₄—X₅—X₆—(Xₐₐ)₁—X₇-M, wherein N=0 to 53, M=0 to 26, and wherein said compound binds preferentially to Bax subfamily member proteins.

### DESCRIPTION OF DRAWINGS

The file of this patent contains at least one photograph or drawing executed in color. Copies of this patent with color drawing(s) or photograph(s) will be provided by the Patent and Trademark Office upon request and payment of necessary fee.

For a fuller understanding of the nature and advantages of the present invention, reference should be had to the following detailed description taken in connection with the accompanying drawings, in which:

FIG. 1 illustrates the structural similarity among Bcl-2 protein complexes. (A) Superimposition of Bcl-xL and Bim complex and CED-9 and EGL-1 complex. (B) Superimposition of Bax, Bcl-xL and Bim complex and CED-9 and EGL-1 complex.

FIG. 2 illustrates the structure-based sequence alignment of Bcl-2 family proteins. (A) Structure-based sequence alignment of the BH3 region from 16 Bcl-2 family proteins. (B) Structure based sequence alignment of the structural elements from 8 Bcl-2 family proteins that can potentially bind to the BH3 region of other Bcl-2 family proteins.

FIG. 3 illustrates interactions of the seven important amino acid residues 1-7 of the Bim (A, C, E, G, I, K, and M) and EGL-1 (B, D, F, H, J, L, and N) with their binding sites in Bcl-xL and CED-9.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to a “pro-apoptotic protein” is a reference to one or more pro-apoptotic proteins and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

The terms “mimetic”, “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics as further described below. Knowing these precise structural features of the naturally-occurring CED-9 in complex with EGL-1, it is advantageous, and well within the level of skill in the art, to design peptidomimetics that have an equivalent structure or function. Such mimetics are another feature of the present invention.

As used herein, the term “therapeutic” means an agent utilized to treat, combat, ameliorate, prevent or improve an unwanted condition or disease of a patient. In part, embodiments of the present invention are directed to promote apoptosis in cells that make up diseased or damaged tissues.

A “therapeutically effective amount” or “effective amount” of a composition is a predetermined amount calculated to achieve the desired effect, i.e., to effectively modulate the activity of a Bcl-2 family member protein. A therapeutically effective amount of a composition that modulates the activity of CED-9 (or other anti-apoptotic Bcl-2 family members such as Bcl-2 or Bcl-xL) is typically an amount such that when it is administered in a physiologically tolerable excipient composition, it is sufficient to achieve the desired effect. Effective amounts of compounds of the present invention can be measured by increased apoptosis. In a preferred embodiment, a therapeutically effective amount refers to the agents ability to preferentially bind to the target, including for example Bcl-2, Bcl-xL, or Bax.

Over-expression of Bcl-2 and/or Bcl-xL is involved in several forms of human cancer. Since these proteins are known to sequester caspase activating proteins that are necessary for the induction of apoptosis, the ability to inhibit this activity in Bcl-2 family proteins will allow for the activation of apoptosis in diseased cells and the elimination of the cancerous tissue. Several anti-cancer therapies depend on the use of pro-apoptotic agents such as radiation and chemo-therapy which are non-specific and potentially toxic to patients. The ability to specifically target proteins that enhance apoptosis will allow for the development and use of anti-cancer therapies that are more specific and less toxic then the traditional therapeutic regimen.

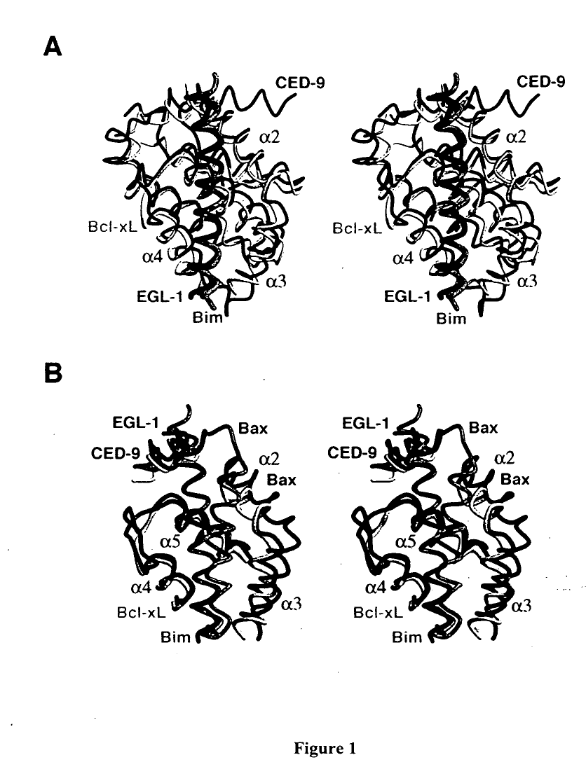

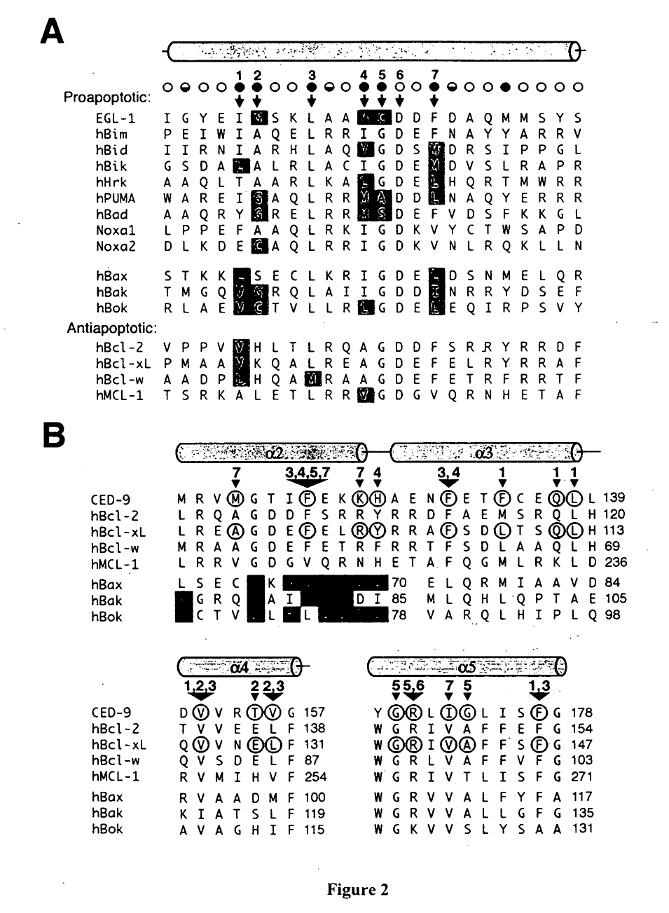

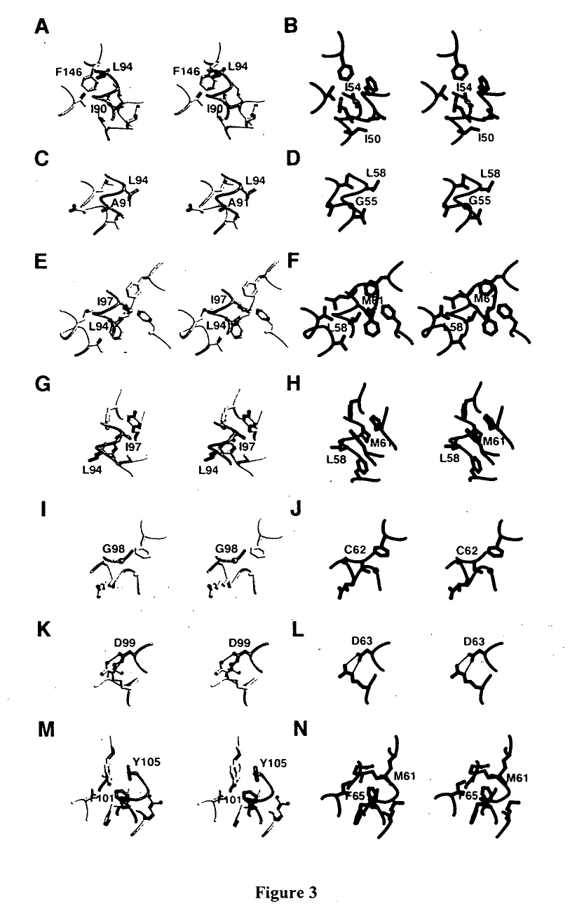

Presently, the three-dimensional crystal structure of CED-9, a Bcl-2 homologue, in complex with a functional EGL-1 fragment encompassing the BH-3 region at 2.2 Å resolution has been characterized as reported in Yan et al., Structural, Biochemical, and Functional Analyses of CED-9 Recognition by the Proapoptotic Proteins EGL-1 and CED-4, 15 MOL. CELL., 1-8, (2004), herein incorporated by reference in its entirety. Previous structures of Bcl-2 family protein complexes involve the same receptor, Bcl-xL, and similar ligands, a BH-3 peptide from Bak, Bad, or Bim. Because Bcl-xL was the only receptor for the BH3 Only proteins in these previous structures and because the binding interactions showed some degrees of variability in these structures, our previous knowledge did not allow derivation of any general principles that govern interactions among the Bcl-2 family proteins. In this regard, the crystal structure of CED-9 bound to EGL-1 significantly expands our knowledge base on which such general principles can be contemplated. Most surprisingly, despite a limited sequence homology between CED-9 and Bcl-xL, the way EGL-1 interacts with CED-9 appears to be highly analogous to the way Bim interacts with Bcl-xL (FIGS. 1 and 3). This observation indicates that such general principles, which are outlined in FIGS. 2 and 3, do exist. Thus, because of the structural similarity and structure based sequence similarity among Bcl-2 family members, the crystal structure of CED-9 in complex with EGL-1 allows for comparison with a homologous but distinct ligands and the Bcl-2 family members receptors and represents an important step in the rationalization of the principles governing the interactions among all Bcl-2 family proteins. As depicted in FIG. 1, the three-dimensionally rendered images of the crystal structures of Bcl-xL/Bim and CED-9/EGL-1 complexes are superimposable on one another. Further, as shown in FIG. 1B, the crystal structure of Bax binding it's C-terminal tail superimposes well with both the Bcl-xL/Bim and CED-9/EGL-1 complex structures. FIGS. 2A and 2B show the structurally based amino acid sequence alignments of EGL-1 and CED-9 and several human homologs. Understanding the principles that govern the interactions among the Bcl-2 family proteins will allow for the rational design of compounds that can discriminate specifically against Bax family, such as but not limited to Bax and Bak, while retaining specific and high-affinity binding to Bcl-2 family members, such as but not limited to Bcl-xL, CED-9, and Bcl-2. The rational design of compounds and improved specificity will allow these compounds to be used in treating diseases involving inhibition of apoptosis while minimizing potential side effects.

The EGL-1/CED-9 complex adopts a compact, globular fold, resembling a single folding unit. CED-9 comprises 7 α helices, with the central hydrophobic helix α5 surrounded by 6 helices and several surface loops. A 27-amino acid fragment of the EGL-1 protein (residues 47-73) forms a single amphipathic α helix, packing against CED-9 helices α2, α3, α4, α5, and α7 over an extended hydrophobic surface cleft. Residues N- or C-terminal to the EGL-1 helix were disordered in the crystals and do not appear to be involved in binding to CED-9. The EGL-1 fragment (residues 48-72) binds to CED-9 (residues 68-251) with a dissociation constant of 6.4 nM, nearly identical to that of the full-length EGL-1 protein to CED-9 (6.7 nM).

Compared to the free CED-9 protein, EGL-1-bound CED-9 undergoes significant structural rearrangements leading to the exposure of an extended hydrophobic surface cleft. Non-polar residues from the amphipathic EGL-1 helix interact with this hydrophobic surface cleft on CED-9. This interaction results in the burial of 2454 Å² exposed surface area. To configure this surface cleft, residues on helix α4 of CED-9 are translocated over a distance of 8-12 Å. This unusual conformational flexibility may underlie the critical functions of CED-9 and other Bcl-2 family proteins.

The driving force for the binding of EGL-1 to CED-9 appears to be van der Waals interactions. Nine hydrophobic side chains as well as two glycine residues (Gly51 & Gly55) from the amphipathic EGL-1 helix make extensive contacts to the hydrophobic surface cleft on CED-9. At the N-terminal portion of the EGL-1 helix, two isoleucines (Ile50 & Ile54) and two glycines (Gly51 & Gly55) stack against the wedge between helices α3 and α4 of CED-9. At the center of the interface, Phe65 of EGL-1 is nestled in a hydrophobic pocket formed by CED-9 residues Met119, Phe123, Lys126, Ile 172, and Met231, whereas Met61 of EGL-1 interacts with Phe123, Phe131, Phe134, and His127 of CED-9. At the C-terminal portion of the EGL-1 helix, there are 6 inter-molecular hydrogen bonds in addition to van der Waals contacts between Met69/Met70 of EGL-1 and surrounding CED-9 residues. In particular, the carboxylate side chains of Asp63 and Asp66 make a pair of charge-stabilized hydrogen bonds to Arg170 and Arg219 of CED-9, respectively.

Seven conserved amino acids, Ile54, Gly55, Leu58, Met61, Cys62, Asp63, and Phe65, interact directly with the conserved binding pocket of CED-9 and appear to be necessary for EGL-1 binding to CED-9 and CED-9 mediated apoptosis. These seven amino acids are conserved between Bim and EGL-1 (FIG. 2). FIG. 3 shows the conservation of specific contacts between these seven amino acids at the Bcl-xL/Bim and CED-9/EGL-1 interfaces. It should be noted that Ile54, Gly55, Leu58, and Met61 utilize contacts with CED-9 amino acids within the surface binding cleft that are conserved among Bcl-2 and Bax proteins including but not limited to Bcl-2, Bcl-xL, Bax and Bak. However, EGL-1 amino acid residues Cys62, Asp63 and Phe 65 bind to CED-9 utilizing contacts with amino acids that are divergent between Bcl-2 and Bax family members (FIG. 2B, accession codes 1F16, 1PQ1, and 1TY4 in www.rcsb.org). Therefore, the ability of a BH-3 Only protein to discriminate between Bcl-2 and Bax must be conferred by the affinity of these proteins for the region of the binding cleft where Cys62, Asp63, and Phe65 mediated contacts are made.

The identification of the EGL-1 binding cleft on CED-9 and the specific residues that make up said cleft from the structural data provided herein will provide the basis for molecular modeling and the design of agents that bind to Bcl-2 family member proteins, but not Bax family member proteins. Such basis is made clear by the structure-based comparison between the Bcl-2/Bcl-xL subfamily and the Bax/Bak subfamily, which show different patterns of amino acids in the binding sites for the seven conserved amino acids from the BH3 Only proteins. Such differences cannot de deduced from prior knowledge and are made available after the crystal structure of the CED-9/EGL-1 complex, which supports the existence of general principles that govern the interactions among the Bcl-2 family of proteins.

Programmed cell death in C. elegans is initiated by the binding of EGL-1 to CED-9, which disrupts the CED-4/CED-9 complex allowing CED-4 to activate the cell-killing caspase CED-3. The C-terminal half of EGL-1 (amino acids 45-87) is necessary and sufficient for binding to CED-9 and the induction of apoptosis. The structure of the EGL-1/CED-9 complex revealed that EGL-1 adopts an extended α-helical conformation and binds to a hydrophobic surface cleft of CED-9 that is absent in the free CED-9 protein. This cleft is only formed after major structural rearrangements induced by EGL-1 binding. These structural rearrangements primarily involve the α4 helix of CED-9 and the loop immediately following α4 helix. A surface patch on CED-9, different from that required for binding to EGL-1, was identified to be responsible for binding to CED-4, and the CED-4 binding element has been mapped to the loop following α4 helix. This observation indicates that the binding of EGL-1 to CED-9 will inevitably induce a rearrangement of the loop following helix α4 and thus destabilize the binding of CED-4 to CED-9, leading to the release of CED-4 from the CED-4/CED-9 complex.

These data and analysis strongly suggest a model by which EGL-1 displaces CED-4 from the CED-4/CED-9 complex. In this model, CED-4 binds to a surface area of CED-9 that is next to the EGL-1-binding element. This interaction allows CED-9 to sequester CED-4 to the outer membrane of mitochondria. At the onset of programmed cell death, EGL-1 is expressed and binds to CED-9 inducing conformational changes in the α4-loop region of CED-9 resulting in the disruption of the CED-4/CED-9 complex, because the specific conformation of EGL-1-bound CED-9 is not compatible with that required for binding to CED-4.

Embodiments of the invention provide methods for the design of compounds that can discriminate between pro-apoptotic Bcl-2 family members (Bax and Bak, for example) and anti-apoptotic Bcl-2 family members (Bcl-2 and Bcl-xL, for example).

In one embodiment the method comprises identifying agents that specifically bind to Bcl-2 by comparing the binding of the agent to Bcl-2 and Bax. These methods are well known to those of skill in the art. For example, testing the efficacy of an agent can be performed by analyzing the ability of the test compound to bind purified Bcl-2 and Bax directly.

In another embodiment, the ability of the agent to induce apoptosis is tested. Apoptosis can be detected by observing cellular changes such as cell shrinkage, DNA degradation, collapse of cells into small apoptotic bodies, etc.

Various techniques including computational analysis, similarity mapping and the like can all be used in this modeling process. See e.g., Perry et al., in OSAR: Quantitative Structure-Activity Relationships in Drug Design, pp. 189-193, Alan R. Liss, Inc., 1989; Rotivinen et al., Acta Pharmaceutical Fennica, 97:159-166 (1988); Lewis et al., Proc. R. Soc. Lond., 236:125-140 (1989); McKinaly et al., Annu. Rev. Pharmacol. Toxiciol., 29:111-122 (1989). Commercial molecular modeling systems available from Polygen Corporation, Waltham, Mass., include the CHARMm program, which performs the energy minimization and molecular dynamics functions, and QUANTA program which performs the construction, graphic modeling and analysis of molecular structure. Such programs allow interactive construction, visualization and modification of molecules. Other computer modeling programs are also available from BioDesign, Inc. (Pasadena, Calif.), Hypercube, Inc. (Cambridge, Ontario), and Allelix, Inc. (Mississauga, Ontario, Canada). From these analyses the desired agents can be formulated and designed.

In one embodiment of the invention, an agent that specifically binds Bcl-2 is designed by generating and graphically displaying the three-dimensional structure of the CED-9/EGL-1 binding site, creating compounds with a spatial structure complimentary to the CED-9/EGL-1 binding site, testing the compound that function in promoting apoptosis, and selecting for compounds that promote apoptosis.

In another embodiment, the desired agent has binding groups that correspond to (i.e., have similar binding characteristics) as the binding groups as EGL-1.

In another embodiment, the desired agent contains binding groups that do not correspond to the binding groups of EGL-1.

In a further embodiment, an agent that specifically neutralizes Bcl-2 is designed by obtaining the three-dimensional structure of the candidate agent, obtaining sets of atomic coordinates for the BH-3 binding domains of Bcl-2 and Bax, employing a computer-aided molecular modeling program to combine the atomic coordinates of the candidate agents with the BH-3 binding domains of Bcl-2 and Bax to create a three-dimensional models of the complexes of the candidate agent with either Bcl-2 or Bax, performing a fitting operation to quantify association between the candidate agent to the BH-3 binding domains of Bcl-2 and Bax, selecting candidate agents who associate preferentially with Bcl-2, and testing the selected agents for the ability to promote apoptosis.

In another embodiment, the method described above is utilized to create three-dimensional models of the candidate agents with either Bcl-2 or Bax, these models are displayed graphically, and the graphically displayed structures are visually inspected to evaluate the candidate agents ability to bind preferentially to Bcl-2.

A variety of techniques are available for constructing peptide mimetics with the same or similar desired biological activity as the corresponding native but with more favorable activity than the peptide with respect to solubility, stability, cell permeability, and/or susceptibility to hydrolysis or proteolysis (see, e.g., Morgan & Gainor, Ann. Rep. Med. Chem. 24, 243-252, 1989). Certain peptidomimetic compounds are based upon the amino acid sequence of the peptides of the invention. Often, peptidomimetic compounds are synthetic compounds having a three-dimensional structure (i.e. a “peptide motif”) based upon the three-dimensional structure of a selected peptide. The peptide motif provides the peptidomimetic compound with the desired biological activity, i.e., binding to Bcl-2 family proteins, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, stability to radiological elements, greater affinity and/or avidity and prolonged biological half-life.

Peptidomimetic design strategies are readily available in the art (see, e.g., Ripka & Rich, Curr. Op. Chem. Biol. 2, 441-452, 1998; Hruby et al., Curr. Op. Chem. Biol. 1, 114-119, 1997; Hruby & Balse, Curr. Med. Chem. 9, 945-970, 2000). One class of peptidomimetics a backbone that is partially or completely non-peptide, but mimics the peptide backbone atom—for atom and comprises side groups that likewise mimic the functionality of the side groups of the native amino acid residues. Several types of chemical bonds, e.g. ester, thioester, thioamide, retroamide, reduced carbonyl, dimethylene and ketomethylene bonds, are known in the art to be generally useful substitutes for peptide bonds in the construction of peptidomimetics. Another class of peptidomimetics comprises a small non-peptide molecule that binds to another peptide or protein, but which is not necessarily a structural mimetic of the native peptide. Yet another class of peptidomimetics has arisen from combinatorial chemistry and the generation of massive chemical libraries. These generally comprise novel templates which, though structurally unrelated to the native peptide, possess necessary functional groups positioned on a nonpeptide scaffold to serve as “topographical” mimetics of the original peptide (Ripka & Rich, 1998, supra).

Peptide components of this invention preferably include the 20 naturally-occurring amino acids. However, incorporation of known artificial amino acids such as beta or gamma amino acids and those containing non-natural side chains, and/or other similar monomers such as hydroxyacids are also contemplated, with the effect that the corresponding peptide is not completely inhibited from binding Bcl-2 family proteins, preferably binding CED-9 and being permeable to the cell.

The functional agents can act as pro-apoptotic ligand agonists for specific members of the Bcl-2 family, such as Bcl-2 and Bcl-xL, while discriminating against members such as Bax and Bak and thus be able to mediate the same biochemical and pharmacological effects (e.g. binding to, and inhibiting the function of, the anti-apoptotic members of the Bcl-2 family for the promotion of apoptosis). The analogs identified can be used in therapeutic applications where apoptosis is desired, such as the treatment of cancer. Agents which may not be appropriate for therapeutic use, can be used to further design effective therapeutic compounds.

In another embodiment, a template can be formed based on the established model for EGL-1 binding to CED-9. Various agents can be designed by linking various chemical groups or moieties to the template. Various moieties of the template can also be replaced. In some embodiments of the invention the peptide or mimetics thereof may be cyclized, e.g., by linking the N-terminus and C-terminus together, to increase its stability. These rationally designed compounds are further tested. In this manner, pharmacologically acceptable and stable compounds with improved efficacy and reduced side-effects can be developed. The compounds identified in accordance with the present invention can be incorporated into a pharmaceutical formulation suitable for administration to an individual.

In one embodiment, unoccupied (aqueous) space between the van der Waals surface of the ligand and the surface defined by residues in the binding site of the receptor may be used to identify gaps in atom-atom contact representing volume that could be occupied by new functional groups on a modified version of the lead compound. More efficient use of the unoccupied space in the binding site could lead to a stronger binding compound if the overall energy of such a change is favorable. A region of the binding pocket which has unoccupied volume large enough to accommodate the volume of a group equal to or larger than a covalently bonded carbon atom, for example, can be identified as a promising position for functional group substitution. Functional group substitution at this region can constitute substituting something other than a carbon atom, such as oxygen. If the volume is large enough to accommodate a group larger than a carbon atom, a different functional group which would have a high likelihood of interacting with protein residues in this region may be chosen. Features which contribute to interaction with protein residues and identification of promising substitutions include hydrophobicity, size, rigidity and polarity. The combination of docking, Kᵢ estimation, and visual representation of sterically allowed room for improvement permits prediction of potent derivatives. The derivatives may be further tested for their efficacy in promoting apoptosis.

In a preferred embodiment of the invention, the binding affinity of the mimetic is at least about 6.0 nM, more preferably at least about 6.4 nM. In another preferred embodiment the binding affinity of the mimetic is at least about 6.7 nM.

The mimetics are preferably administered in effective amounts. An effective amount is that amount of a preparation that alone, or together with further doses, produces the desired response. This may involve only slowing the progression of the disease temporarily, although preferably, it involves halting the progression of the disease permanently or delaying the onset of or preventing the disease or condition from occurring. This can be monitored by routine methods. Generally, doses of active compounds would be from about 0.01 mg/kg per day to 1000 mg/kg per day. It is expected that doses ranging from 50-500 mg/kg will be suitable, preferably intravenously, intramuscularly, or intradermally, and in one or several administrations per day.

In general, routine experimentation in clinical trials will determine specific ranges for optimal therapeutic effect for each therapeutic agent and each administrative protocol, and administration to specific patients will be adjusted to within effective and safe ranges depending on the patient condition and responsiveness to initial administrations. However, the ultimate administration protocol will be regulated according to the judgment of the attending clinician considering such factors as age, condition and size of the patient, the ligand/compound potencies, the duration of the treatment and the severity of the disease being treated. For example, a dosage regimen of the ligand/compound can be oral administration of from 1 mg to 2000 mg/day, preferably 1 to 1000 mg/day, more preferably 50 to 600 mg/day, in two to four (preferably two) divided doses, to reduce tumor growth. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient tolerance permits. Multiple doses per day are contemplated to achieve appropriate systemic levels of compounds. Generally, a maximum dose is used, that is, the highest safe dose according to sound medical judgment. Those of ordinary skill in the art will understand, however, that a patient may insist upon a lower dose or tolerable dose for medical reasons, psychological reasons or for virtually any other reason.

A variety of administration routes are available. The particular mode selected will depend, of course, upon the particular chemotherapeutic drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include, but are not limited to, oral, rectal, topical, nasal, intradermal, inhalation, intra-peritoneal, or parenteral routes. The term “parenteral” includes subcutaneous, intravenous, intramuscular, or infusion. Intravenous or intramuscular routes are particularly suitable for purposes of the present invention.

In one aspect of the invention, a ligand/compound as described herein, does not adversely affect normal tissues, while sensitizing tumor cells to the additional chemotherapeutic/radiation protocols. While not wishing to be bound by theory, it would appear that because of this tumor specific induced apoptosis, marked and adverse side effects such as inappropriate vasodilation or shock are minimized. Preferably, the composition or method is designed to allow promote programmed cell death.

Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be desirable. Long-term release, are used herein, means that the implant is constructed and arranged to deliver therapeutic levels of the active ingredient for at least 30 days, and preferably 60 days. Long-term sustained release implants are well-known to those of ordinary skill in the art.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride, chlorobutanol, parabens and thimerosal.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous preparation of a ligand/compound, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa. which is incorporated herein in its entirety by reference thereto.

This invention and embodiments illustrating the method and materials used may be further understood by reference to the following non-limiting examples.

### EXAMPLE 1

This example describes the characterization of EGL-1/CED-9 interactions. To determine the mechanism of CED-9 recognition by EGL-1, the EGL-1/CED-9 interaction was characterized. Various fragments of the GST-EGL-1 protein were evaluated for their abilities to interact with the CED-9 protein (residues 1-251). The N-terminal half (residues 1-45) of EGL-1 did not show detectable binding to CED-9. In contrast, the C-terminal half (residues 45-87 or 45-91) formed a stable complex with CED-9. Using a similar strategy, residues 68-237 of CED-9 were found to be the minimal structural core that is necessary and sufficient for binding to EGL-1. Removal of four hydrophobic residues (F₈₈F₈₉A₉₀F₉₁ ) at the C-terminus of EGL-1 did not affect its binding to CED-9, but significantly improved expression levels of the recombinant proteins.

To further characterize EGL-1/CED-9 interactions, a number of missense mutations were introduced into EGL-1 and the ability of the resulting EGL-1 mutants to interact with CED-9 was determined. The mutation of two residues in EGL-1, G55E and F65A which reside in the BH-3 domain of EGL-1, completely abolished its interaction with CED-9, suggesting a critical role of these two residues and potentially the BH-3 domain in mediating EGL-1/CED-9 interaction.

### EXAMPLE 2

This example demonstrates the structure of an EGL-1/CED-9 complex. To reveal the mechanism of CED-9 recognition by EGL-1, CED-9 (residues 68-237) in complex with EGL-1 (residues 45-87 or 31-87) were crystallized. Crystals were obtained after extensive effort involving more than 100 different EGL-1/CED-9 complexes over 30,000 crystallization conditions. The structure was determined by molecular replacement and refined to 2.2 Å resolution.

The EGL-1/CED-9 complex adopts a compact, globular fold, resembling a single folding unit. CED-9 comprises 7 α helices, with the central hydrophobic helix α5 surrounded by 6 helices and several surface loops. A 27-amino acid fragment of the EGL-1 protein (residues 47-73) forms a single amphipathic α helix, packing against CED-9 helices α2, α3, α4, α5, and α7 over an extended hydrophobic surface cleft. Residues N- or C-terminal to the EGL-1 helix were disordered in the crystals and thus are not involved in binding to CED-9. To confirm that these regions of EGL-1 do not contribute to CED-9 binding, the binding affinities between CED-9 and various fragments of EGL-1 were examined using isothermal titration calorimetry (ITC). The results revealed that the EGL-1 fragment (residues 48-72) binds to CED-9 (residues 68-251) with a dissociation constant of 6.4 nM, nearly identical to that of the full-length EGL-1 protein to CED-9 (6.7 nM).

Compared to the free CED-9 protein, EGL-1-bound CED-9 undergoes significant structural rearrangements, leading to the exposure of an extended hydrophobic surface cleft. Non-polar residues from the amphipathic EGL-1 helix interact with this hydrophobic surface cleft on CED-9. This interaction results in the burial of 2454 Å² exposed surface area. To configure this surface cleft, residues on helix α4 of CED-9 are translocated over a distance of 8-12 Å. This unusual conforrnational flexibility might underlie the critical functions of CED-9 and other Bcl-2 family proteins.

The conformational change observed in the EGL-1/CED-9 complex is quite different from that reported in the mammalian complexes involving Bcl-xL. In those cases, the change primarily involves a lateral movement of helix α3, such as that in the Bak-BH-3/Bcl-xL complex. However, in the EGL-1/CED-9 complex, helix α3 remains nearly unchanged before and after EGL-1 binding; yet helix α4 undergoes drastic structural rearrangements. In addition, the EGL-1/CED-9 interface is more extensive than that of the reported mammalian complexes involving Bcl-xL and a BH3 peptide, as judged by the number of van der Waals contacts and the extent of buried surface area.

### EXAMPLE 3

This example describes the EGL-1/CED-9 interface. The driving force for the binding of EGL-1 to CED-9 is van der Waals interactions. Nine hydrophobic side chains as well as two glycine residues (Gly51 & Gly55) from the amphipathic EGL-1 helix make extensive contacts to the hydrophobic surface cleft on CED-9. At the N-terminal portion of the EGL-1 helix, two isoleucines (Ile50 & Ile54) and two glycines (Gly51 & Gly55) stack against the wedge between helices α3 and α4 of CED-9. At the center of the interface, Phe65 of EGL-1 is nestled in a hydrophobic pocket formed by CED-9 residues Met119, Phe123, Lys126, Ile 172, and Met231 whereas Met61 of EGL-1 interacts with Phe123, Phe131, Phe134, and His127 of CED-9. At the C-terminal portion of the EGL-1 helix, there are 6 inter-molecular hydrogen bonds in addition to van der Waals contacts between Met69/Met70 of EGL-1 and surrounding CED-9 residues. In particular, the carboxylate side chains of Asp63 and Asp66 make a pair of charge-stabilized hydrogen bonds to Arg170 and Arg219 of CED-9, respectively.

Seven conserved amino acids, Ile54, Gly55, Leu58, Met61, Cys62, Asp63, and Phe65, interact directly with the conserved binding pocket of CED-9 and appear to be necessary for EGL-1 binding to CED-9 and CED-9 mediated apoptosis and are conserved between Bim and EGL-1 (FIG. 2). FIG. 3 shows the conservation of specific contacts between these seven amino acids at the Bcl-xL/Bim and CED-9/EGL-1 interfaces. It should be noted that Ile54, Gly55, Leu58, and Met61 utilize contacts with CED-9 amino acids within the surface binding cleft that are conserved among Bcl-2 and Bax proteins including but not limited to Bcl-2, Bcl-xL, Bax and Bak. However, EGL-1 amino acid residues Cys62, Asp63 and Phe 65 bind to CED-9 utilizing contacts with amino acids that are divergent between Bcl-2 and Bax family members (FIG. 2B). Therefore, the ability of a BH-3 Only protein to discriminate between Bcl-2 and Bax can be conferred by the affinity of these proteins for the region of the binding cleft where Cys62, Asp63, and Phe65 mediated contacts are made.

The key residues of EGL-1 that interact with CED-9 are highly conserved. Interestingly, residues from the predicted BH-3 domain (Leu58-Asp66) only contribute to approximately half of the observed interactions and one third of the extended EGL-1 helix. This structural finding nicely explains the observation that the EGL-1 BH-3 peptide does not stabilize the CED-9 protein to the same extent as the intact EGL-1 protein.

### EXAMPLE 4

This example describes the biochemical and functional analysis of EGL-1/CED-9 interactions. To corroborate the structural analysis, mutations on the interface residues of EGL-1 were examined for their potential to weaken or disrupt its binding to CED-9. Various mutant EGL-1 fragments were purified as fusion proteins with glutathione S-transferase (GST) and their interactions with CED-9 were investigated using a GST-mediated pull-down assay. Consistent with the observed structural features, substitution of a bulky hydrophobic residue (Leu58, Met61, Phe65, or Met69) by Ala in EGL-1 did not significantly affect its interaction with CED-9. Strikingly, substitution of Gly55 by a negatively charged glutamate residue was not sufficient to abolish interaction with CED-9. Nonetheless, a double mutant (mut2, G55E/F65A) and a quadruple mutant (mut4, G55E/L58A/F65A/M69A) failed to bind to CED-9. These results are consistent with the structural observation that the intimate interface between EGL-1 and CED-9 closely resembles the interior of a single folded protein and is thus relatively resistant to single missense mutations.

EGL-1 induces apoptosis by displacing CED-4 from the CED-4/CED-9 complex. To recapitulate this finding in vitro, a CED-4 displacement assay was designed in which pre-assembled CED-4/CED-9 complex was immobilized on glutathione resin and was then challenged with various EGL-1 fragments. After extensive washing, the remaining CED-4/CED-9 complex was eluted from the resin and visualized on SDS polyacrylamide gel. As anticipated, the wild-type EGL-1 protein (residues 1-87) or the C-terminal fragment of EGL-1 (45-87) completely displaced CED-4 from the CED-4/CED-9 complex. In contrast, most missense mutations at the EGL-1/CED-9 interface weakened the ability of EGL-1 to displace CED-4 from the CED-4/CED-9 complex. Notably, the double mutant (mut2, G55E/F65A) and quadruple mutant (mut4, G55E/L58A/F65A/M69A) completely failed to disrupt the CED-4/CED-9 complex. Interestingly, some EGL-1 mutants, such as G55E and L58A, while retaining their ability to bind to CED-9, exhibited significantly reduced ability in displacing CED-4 from the CED-4/CED-9 complex in vitro. Hence the ability of an EGL-1 mutant to disrupt the CED-4/CED-9 complex provides a sensitive and biologically meaningful evaluation of the effect of the EGL-1 missense mutation on its function. The in vitro studies are in complete agreement with the structural observation.

### EXAMPLE 5

In this example, the ability of EGL-1 mutants to disrupt the CED-4/CED-9 complex was tested. EGL-1 mutants that are unable to disrupt the CED-4/CED-9 complex should exhibit decreased ability to induce cell death compared to the WT EGL-1 protein in vivo. To examine this scenario, constructs were injected into ced-1 (e1735); egl-1 (n1084 n3082) animals that direct expression of various EGL-1 fragments under the control of the C. elegans heat-shock promoters. Cell corpses were scored in the anterior head region of four-fold transgenic embryos after the heat-shock treatment. Few cell corpses were observed in the ced-1 (e1735); egl-1 (n1084 n3082) embryos because the egl-1 (n1084 n3082) mutation blocks almost all somatic cell deaths. As shown previously, expression of the full-length EGL-1 protein induced robust cell killing. Expression of the EGL-1 C-terminal fragment (residues 46-87) induced about half the number of cell corpses as the full-length EGL-1, indicating that this EGL-1 fragment is functional in vivo. In contrast, expression of the EGL-1 N-terminal fragment (residues 1-45) did not induce cell death, consistent with an earlier observation.

The cell-killing activity of various EGL-1 mutants correlated extremely well with their in vitro biochemical activities in binding to CED-9 and in displacing CED-4 from the CED-4/CED-9 complex. For example in vitro, the EGL-1 double mutant (G55E/F65A) failed to bind to CED-9 or to displace CED-4 from the CED-4/CED-9 complex, whereas the EGL-1 (G55E) mutant retained its ability to interact with CED-9 yet exhibited a decreased ability to disrupt the CED-4/CED-9 complex. In vivo, EGL-1 (G55E/F65A) induced no cell killing while EGL-1 (G55E) induced cell death but at a significantly lower level than that of the wild-type EGL-1 protein.

### EXAMPLE 6

This example describes the biochemical analysis of CED-4/CED-9 interactions. To further define the molecular mechanisms by which EGL-1 induces the release of CED-4 from CED-9-mediated sequestration, the identify of surface residues in CED-9 that are important for binding to CED-4 was analyzed. It was hypothesized that for molecular recognition to occur, at least some of the CED-9 residues that are important for binding to CED-4 must be solvent-exposed prior to binding. Therefore, the structure of CED-9 in isolation was examined and a total of 44 amino acids were identified, each with at least 30% of its surface area exposed to solvent. These 44 amino acids were grouped based on primary sequences and 20 CED-9 mutant clones were generated, each containing 1-5 missense mutations that together covering all 44 solvent-exposed residues. All 20 CED-9 proteins were cloned into an expression vector and over-expressed in E. coli and purified to homogeneity.

Next, the abilities of these CED-9 mutants to interact with CED-4 was examined. As anticipated, most mutations exhibited no apparent effect on the interaction between CED-9 and CED-4. In contrast, three CED-9 mutants, F146N, N158A/A159G/Q160A and R211E/N212G exhibited significantly weakened interactions with CED-4. Interestingly although these mutations affect residues widely separated in the primary sequence, they all map to the same surface area on CED-9, defining a surface patch important for CED-4 binding. This surface patch may constitute the primary CED-4-binding motif. None of the other 16 mutations, which cover the entirety of the CED-9 surface, affected the CED-4/CED-9.

In conclusion, the combined structural, biochemical, and functional studies have identified the molecular mechanisms by which EGL-1 binds to CED-9 and induces the release of CED-4 from the CED-4/CED-9 complex. These studies define a mechanistic framework for understanding apoptosis activation in C. elegans.

Although the present invention has been described in considerable detail with reference to certain preferred embodiments thereof, other versions are possible. Therefore the spirit and scope of the appended claims should not be limited to the description and the preferred versions contain within this specification.

What is claimed is:

1. A method of identifying agents that promote apoptosis by selectively interacting with Bcl-2 subfamily member proteins comprising:
observing binding of an agent with a Bcl-2 subfamily member protein selected from the group consisting of Bcl-2 and Bcl-xL;
observing binding of an agent with a Bax subfamily member protein selected from the group consisting of Bax and Bak; and
selecting an agent based upon preferential binding with Bcl-2 subfamily member proteins.

2. The method of claim 1, wherein binding of said agent mimics the binding of the C-terminal 45 amino acids of EGL-1 to CED-9.

3. The method of claim 1, wherein binding of said agent mimics the binding of EGL-1 to CED-9 at amino acid residues 54, 55, 58, 61, 62, 63, and 65.

4. The method of claim 1, wherein binding of said agent mimics the binding of the BH-3 domain of EGL-1 to CED-9.

5. The method of claim 4, wherein binding of said agent mimics the binding of the EGL-1 to CED-9 at amino acid residues 5, 6, and 7 of the BH-3 domain.

6. A composition comprising the amino acid sequence N—X₁—X₂—(Xₐₐ)₂—X₃—(Xₐₐ)₂—X₄—X₅—X₆—(Xₐₐ)₁—X₇-M and a carrier, wherein N=0 to 53, M=0 to 26, and wherein said compound binds preferentially to Bcl-2 subfamily member proteins.

7. The composition of claim 6, wherein said composition binds at a hydrophobic pocket on the surface of Bcl-2 through interactions between amino acids in said pocket and the side-chains of amino acid residues X₁-X₇ of said composition.

8. The composition of claim 7, wherein said interactions are selected from a group consisting of hydrogen bonds and van der Waals interactions.

9. The composition of claim 7, wherein the interaction at amino acid residue X₆ is hydrogen bonding.

10. The composition of claim 7, wherein the side chains of amino acids X₅, X₆, and X₇ have been modified to fill the aqueous space created by the van der Waals radii of amino acids residues Met119, Phe123, Lys126, Phe133, Gln137, Leu138, Val152, Thr155, Val156, Gly169, Arg170, Gly171, Ile172, Phe177, and Met 231 of CED-9.

11. The composition of claim 9, wherein said composition exhibits a binding affinity for Bcl-2 of at least about 6.0 nM.

12. A method of inducing apoptosis comprising administering a compound that selectively binds to Bcl-2, and wherein said compound does not bind to Bax.

13. The method of claim 12 wherein said compound exhibits a binding affinity for Bcl-2 of at least about 6.0 nM.

14. A method of making a compound that selectively binds to Bcl-2 subfamily member proteins comprising:
constructing a compound that interacts with CED-9, wherein said compound binds to a hydrophobic pocket on the surface of CED-9; and
determining whether the compound promotes apoptosis.

15. The method of claim 14, wherein binding of said compound mimics the binding of the C-terminal 45 amino acids of EGL-1 to CED-9.

16. The method of claim 14, wherein binding of said compound mimics the binding of EGL-1 to CED-9 at amino acids residues 54, 55, 58, 61, 62, 63, and 65.

17. The method of claim 14, wherein said compound fits within the aqueous space created by the van der Waals radii of amino acids residues Met119, Phe123, Lys126, Phe133, Gln137, Leu138, Val152, Thr155, Val156, Gly169, Arg170, Gly171, Ile172, Phe177, and Met 231 of CED-9.

18. The method of claim 14, wherein said compound has a binding affinity for Bcl-2 of at least about 6.0 nM.

19. A method of identifying agents that promote cellular proliferation by selectively interacting with Bax subfamily member proteins comprising:
observing binding of an agent with a Bcl-2 subfamily member protein selected from the group consisting of Bcl-2 and Bcl-xL;
observing binding of an agent with a Bax subfamily member protein selected from the group consisting of Bax and Bak; and
selecting an agent based upon preferential binding with Bax subfamily member proteins.

20. An isolated and purified peptide comprising the amino acid sequence N—X₁—X₂—Xₐₐ)₂—X₃—(Xₐₐ)₂—X₄X₅—X₆—(Xₐₐ)₁—X₇-M, wherein N=0 to 53, M=0 to 26, and wherein said compound binds preferentially to Bcl-2 subfamily member proteins.

21. An isolated and purified peptide comprising the amino acid sequence N—X₁—X₂—(Xₐₐ)₂—X₃—(Xₐₐ)₂—X₄—X₅—X₆—(Xₐₐ)₁—X₇-M, wherein N=0 to 53, M=0 to 26, and wherein said compound binds preferentially to Bax subfamily member proteins.
